(19) United States
(12) Patent Application Publication
(10) Pub. No.: US 2022/0082506 A1
(43) Pub. Date: Mar. 17, 2022

(54) METHOD AND DEVICE FOR NON-INVASIVE ROOT PHENOTYPING

(71) Applicant: Hi Fidelity Genetics, Inc., Durham, NC (US)

(72) Inventors: Matthew MOORE, Durham, NC (US); Jeffrey AGUILAR, Durham, NC (US); Logan JOHNSON, Durham, NC (US)

(73) Assignee: Hi Fidelity Genetics, Inc., Durham, NC (US)

(21) Appl. No.: 17/421,369

(22) PCT Filed: Jan. 9, 2020
(86) PCT No.: PCT/US2020/012900

§ 371 Date: Jul. 7, 2021

Related U.S. Application Data
(60) Provisional application No. 62/790,880, filed on Jan. 10, 2019.

(51) Int. Cl. G01N 21/84; G01N 21/25; G01N 33/00; G01V 8/20; G01N 21/55

(52) U.S. Cl. CPC G01N 21/84; G01N 21/255; G01N 2021/8466; G01V 8/20; G01N 21/55; G01N 33/0098

(57) ABSTRACT

The present invention generally relates to detecting a root of a plant in soil. An exemplary system comprises a support structure configured to be at least partially disposed in the soil; an LED unit affixed to the support structure, wherein the LED unit comprises an emitter and a detector, wherein the emitter is configured to produce a plurality of outgoing light signals, wherein the detector is configured to receive a plurality of returned light signals corresponding to the plurality of outgoing light signals, and wherein each of the plurality of returned light signals comprises at least a portion of the corresponding outgoing light signal reflected from at least one of the soil and the root; and a microprocessor configured to detect a presence of the root based on the plurality of returned light signals.

### CROSS REFERENCE TO RELATED APPLICATIONS

This application claims the priority benefit of U.S. Provisional Application Ser. No. 62/790,880, filed Jan. 10, 2019, which is hereby incorporated by reference in its entirety.

### FIELD OF THE INVENTION

The present disclosure relates generally to non-invasive root phenotyping, and more specifically to computer-enabled systems, devices, and methods for tracking root growth and monitoring root traits over time.

### BACKGROUND

Root system architecture (RSA) describes the spatial arrangement of roots within the soil that is shaped by genetic and enviromnental factors. The RSA impacts plant fitness, crop performance, grain yield, and can influence a plant's drought tolerance and ability to acquire nutrients. For example, studies have shown that modifying a single gene, DEEPER ROOTING 1 (DRO1), in rice changes the root angle without changing the overall length of the root. This slight change in root angle directs the roots downward, which provides the plant with more access to groundwater. As such, the modified rice (e.g., rice with the DRO1 gene) yields 10% less under drought conditions, whereas unmodified rice (e.g, rice without the DRO1 gene) yields 60% less under the same conditions as compared to well-watered conditions.

Root traits rarely have been applied to breeding programs due, in part, to the difficulty in measuring and monitoring root growth in opaque and complex soils. Current techniques either reduce crop yield or interfere with the plants growing cycle. One technique, for example, uproots field-grown plants for a single time-point measurement. Not only is this technique destructive, but the uprooting process changes in situ factors (e.g., removes the soil foundation), which can bias the measurements (e.g., root angle measurements without soil).

A less destructive technique provides a viewing window such as a rhizotron to observe the roots over time. This technique places a transparent barrier in the path of root growth in order to view the roots that grow adjacent the viewing window of the rhizotron camera. This technique interferes with the plant's natural growing cycle, as it intentionally places an obstruction in the natural path of root development.

Real-time monitoring of the RSA during the growing season without interfering with the plant's growing cycle can provide invaluable information that can be used to produce healthier plants and yield a more abundant crop. As such, a challenge exists for improved, non-invasive techniques for monitoring root phenotypes, such as growth rate, length, angle, and the like.

### BRIEF SUMMARY

In some embodiments, an exemplary system for detecting a root of a plant in soil comprises a support structure configured to be at least partially disposed in the soil; an LED unit affixed to the support structure, wherein the LED unit comprises an emitter and a detector, wherein the emitter is configured to produce a plurality of outgoing light signals, wherein the detector is configured to receive a plurality of returned light signals corresponding to the plurality of outgoing light signals, and wherein each of the plurality of returned light signals comprises at least a portion of the corresponding outgoing light signal reflected from at least one of the soil and the root; and a microprocessor configured to detect a presence of the root based on the plurality of returned light signals.

In some embodiments, the system further comprises a signal extractor configured to extract a plurality of digital readings based on the plurality of returned light signals. In some embodiments, the signal extractor comprises a voltage divider, an analog-to-digital converter, or a combination thereof. In some embodiments, detecting the root based on the plurality of returned light signals comprises: determining a difference between a brightness of a first returned signal and a brightness of a second returned signal of the plurality of returned signals. In some embodiments, the LED unit further comprises a partition, wherein the partition is configured to reduce detection of the plurality of outgoing light signals by the detector. In some embodiments, the LED unit further comprises a lens. In some embodiments, the LED unit is selected based on one or more characteristics of the soil, one or more characteristics of the root, or a combination thereof. In some embodiments, the support structure comprises a paddle, wherein the paddle comprises a plurality of LED units affixed thereto. In some embodiments, the plurality of LED units are arranged in a linear configuration. In some embodiments, the plurality of LED units is arranged based on one or more characteristics of the plant. In some embodiments, the system further comprises one or more capacitive sensors for detecting the root of the plant. In some embodiments, information associated with the plurality of returned light signals are transmitted to a remote computer system via a wireless network. In some embodiments, the microprocessor is configured to detect a presence of an invertebrate in the soil based on the plurality of returned light signals In some embodiments, the microprocessor is configured to determine, based on the plurality of returned light signals: a growth rate of the root, an angle of the root, a density of a group of roots, or a combination thereof. In some embodiments, the system further comprises a power supply electrically coupled to the LED unit, wherein the power supply is configured to provide an electrical charge to the LED unit.

In some embodiments, an exemplary method for detecting a root of a plant in soil comprises transmitting, from an emitter disposed in the soil, a plurality of outgoing light signals; receiving, from a detector disposed in the soil, a plurality of returned light signals corresponding to the plurality of outgoing light signals, wherein each of the plurality of returned light signals comprises at least a portion of the corresponding outgoing light signal reflected from at least one of the soil and the root; extracting a plurality of signal responses corresponding to the plurality of returned light signals; and detecting a presence of the root based on the plurality of signal responses. In some embodiments, the plurality of signal responses comprises a plurality of digital readings. In some embodiments, the method uses a system according to any one of the above embodiments.

In some embodiments, an exemplary method for detecting a root of a plant in soil comprises transmitting, from an emitter disposed in the soil, a plurality of outgoing light signals; receiving, from a detector disposed in the soil, a plurality of returned light signals corresponding to the plurality of outgoing light signals, wherein each of the plurality of returned light signals comprises at least a portion of the corresponding outgoing light signal reflected from at least one of the soil and the root; extracting a plurality of signal responses corresponding to the plurality of returned light signals; and detecting a presence of the root based on the plurality of signal responses. In some embodiments, the plurality of signal responses comprises a plurality of digital readings. In some embodiments, the method further comprises transmitting, from a second emitter disposed in the soil, a second plurality of outgoing light signals; receiving, from a second detector disposed in the soil, a second plurality of returned light signals corresponding to the second plurality of outgoing light signals, wherein each of the second plurality of returned light signals comprises at least a portion of the corresponding outgoing light signal reflected from at least one of the soil and the root; extracting a second plurality of signal responses corresponding to the second plurality of returned light signals; detecting a presence of the root based on the second plurality of signal responses; and based on the first and the second plurality of signal responses, determining a growth characteristic of the plant root, wherein the growth characteristic is selected from the group consisting of growth rate, root angle, root length, and root biomass. In some embodiments, the plant is a row crop. In some embodiments, the plant is selected from the group consisting of maize, soybean, rice, wheat, sorghum, tomato, and alfalfa. In some embodiments, the method uses a system according to any one of the above embodiments.

In some embodiments, an exemplary method for detecting a soil organism comprises transmitting, from an emitter disposed in the soil, a plurality of outgoing light signals; receiving, from a detector disposed in the soil, a plurality of returned light signals corresponding to the plurality of outgoing light signals, wherein each of the plurality of returned light signals comprises at least a portion of the corresponding outgoing fiat signal reflected from at least one of the soil and the soil organism; extracting a plurality of signal responses corresponding to the plurality of returned light signals; and detecting a presence of the soil organism based on the plurality of signal responses. In some embodiments, the plurality of signal responses comprises a plurality of digital readings. In some embodiments, the soil organism is a worm or insect. In some embodiments, the soil organism is a corn root worm. In some embodiments, the method uses a system according to any one of the above embodiments.

In some embodiments, an exemplary method for monitoring growth of a root of a plant in soil comprises positioning a plurality of emitters and a plurality of detectors around a soil location, wherein a plant having a root is planted in the soil location; transmitting, from an emitter of the plurality of emitters disposed in the soil, a plurality of outgoing light signals; receiving, from a detector of the plurality of detectors disposed in the soil, a plurality of returned light signals corresponding to the plurality of outgoing light signals, wherein each of the plurality of returned light signals comprises at least a portion of the corresponding outgoing light signal reflected from at least one of the soil and the root; extracting a plurality of signal responses corresponding to the plurality of returned light signals; detecting a presence of the root based on the plurality of signal responses; and determining a growth characteristic of the plant root based on the detected presence of the root. In some embodiments, an exemplary method for monitoring growth of a root of a plant in soil comprises planting a seed in a soil location; positioning a plurality of emitters and a plurality of detectors around the soil location; after the seed has grown into a plant having a root, transmitting, from an emitter of the plurality of emitters disposed in the soil, a plurality of outgoing light signals; receiving, from a detector of the plurality of detectors disposed in the soil, a plurality of returned light signals corresponding to the plurality of outgoing light signals, wherein each of the plurality of returned light signals comprises at least a portion of the corresponding outgoing light signal reflected from at least one of the soil and the root; extracting a plurality of signal responses corresponding to the plurality of returned light signals; detecting a presence of the root based on the plurality of signal responses; and determining a growth characteristic of the plant root based on the detected presence of the root. In some embodiments, the method uses a system according to any one of the above embodiments.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### DESCRIPTION OF THE FIGURES

FIG. 1 depicts an exemplary non-invasive root phenotyping system.

FIG. 2 depicts a plurality of exemplary LED units affixed to a paddle, according to various examples.

FIG. 3 depicts an exemplary process for detecting a root of a plant in the soil, according to various examples.

FIG. 4 depicts an exemplary process for detecting a root of a plant in the soil, according to various examples.

FIG. 5 depicts an exemplary electronic device in accordance with some embodiments.

FIGS. 6A & 6B depict exemplary data related to root growth obtained by a plurality of exemplary LED units in accordance with some embodiments.

### DETAILED DESCRIPTION

The present disclosure provides for a non-invasive root phenotyping system to detect and/or monitor the growth of a plant root. In some embodiments, the electronic device includes a support structure (e.g., a paddle) suitable for insertion into soil (e.g., adjacent to the plant root). The electronic device further includes a plurality of LED units trellised to the support structure. The system can further include a microprocessor, a signal extractor (e.g., voltage divider, analog to digital converter), and/or a power supply (e.g., voltage or current source). Each LED unit can include an emitter and a detector. The emitter is configured to produce a plurality of outgoing light signals, and the detector is configured to receive a plurality of returned light signals corresponding to the plurality of outgoing light signals. By analyzing how characteristics of the returned light signals vary over time and by correlating the returned light signals with the locations of the LED unit(s) that detected the light signals, the microprocessor can obtain rich information about the objects and events in the soil.

The electronic sensors and devices of the present disclosure implement techniques of non-invasive root phenotyping, such as the techniques for monitoring growth of a plant root, techniques for selecting a plant for breeding based on a root growth characteristic, techniques for determining an effect of a plant-microbe interaction on a root growth characteristic, and/or techniques for monitoring a soil organism. These techniques described herein provide for monitoring of plant root growth in situ while the plant is growing, provide for a higher resolution of monitoring of RSA than existing devices (e.g., mini-rhizotron), and provide for a low-cost solution that is suitable for field use with minimal interference to plant growth.

The following description sets forth exemplary methods, parameters, and the like. It should be recognized, however, that such description is not intended as a limitation on the scope of the present disclosure but is instead provided as a description of exemplary embodiments.

Although the following description uses terms “first,” “second,” etc. to describe various elements, these elements should not be limited by the terms. These terms are only used to distinguish one element from another. For example, a first outgoing light signal could be termed a second outgoing light signal, and, similarly, a second outgoing light signal could be termed a first outgoing light signal, without departing from the scope of the various described embodiments. The first outgoing light signal and the second outgoing light signal are both outgoing light signals, but they are not the same outgoing light signal.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

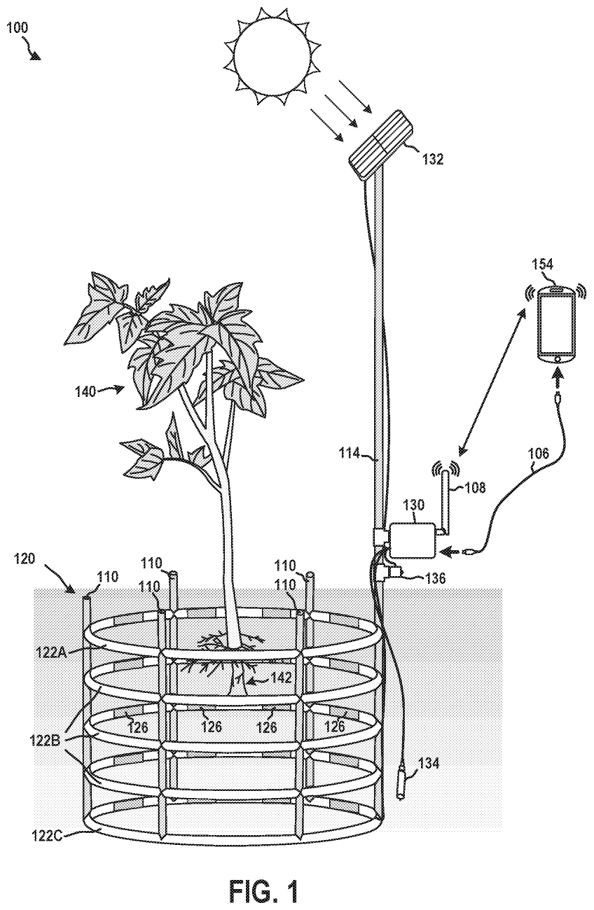

FIG. 1 illustrates an exemplary non-invasive root phenotyping system 100. The root phenotyping system 100 includes a support structure suitable for arrangement in a soil location adjacent a plant 140. The support structure is at least partially disposed in the soil. In the depicted example, the support structure is a cage structure 120 with top circular support 122A, middle circular supports 122B, and bottom circular support 122C, which are connected to extended vertical support 114 and vertical supports 110 that form a backbone for the support structure.

Additional circular supports can be added to a desired cage structure 120. For example, a cage structure can include 1 or more, 2 or more, 3 or more, 4 or more, 5 or more, 6 or more, 7 or more, 8 or more, 9 or more, 10 or more, 11 or more, or 12 or more, etc. circular supports. The number of circular supports to be used can be influenced by, for example, a desired spacing and/or density of the cage circular supports; a size, shape, and/or complexity of the RSA to be monitored; the shape and/or configuration of the device; a number of inputs that may be accommodated by a microcontroller of the present disclosure; and so forth. Likewise, the cage structure 120 can be an auger or include a helical blade affixed to the cage structure 120 to facilitate burrowing the cage structure 120 into the soil around the plant 140,

In some examples, the cage structure 120 is made from any material that resists deformation upon insertion into a desired soil type without affecting the health and growth of the plant 140. For example, the cage structure 120 material can be metal (e.g., galvanized steel, stainless steel), plastic (e.g., bioplastics), and the like. In some examples, the cage structure 120 is made from biodegradable and/or compostable material such as polylactic acid (PLA), poly-3-hydroxybutyrate (PHB), polyhydroxyalkanoates (PHA), and the like. In some instances, a 3-D printer can be utilized to construct the cage structure 120 using a suitable thermoplastic (e.g., PLA). In some instances, the cage structure 120 can be injected molded using a suitable thermoplastic (e.g., PLA).

The cage structure 120 further comprises one or more paddles 126. For example, a plurality of paddles 126 can be trellised to a top circular support 122A, a middle circular support 122B, and a bottom circular support 122C, as depicted in FIG. 1. In some instances, the plurality of paddles 126 can be trellised to the extended vertical support 114 and vertical supports 110 that provide for a relatively fixed position during insertion into a soil location and subsequent operation. In some instances, one or more of the plurality of paddles 126 can be provided on a mesh and positioned between the vertical supports 110 and the circular supports 122A, 122B, 122C.

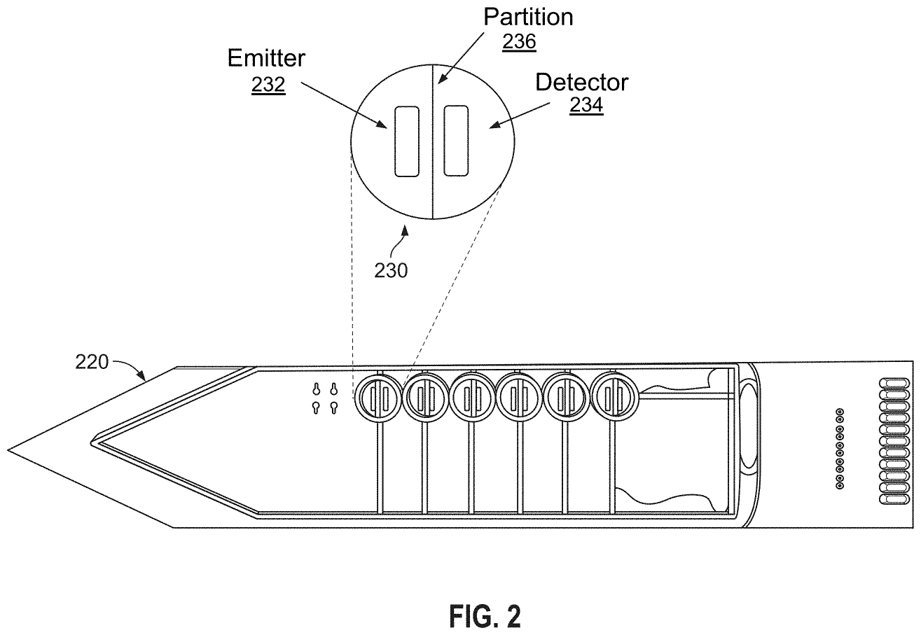

As discussed further with reference to FIGS. 2-4, one or more LED units (not depicted) can be affixed to one or more of the paddles 126. Each of the LED units is electrically coupled (e.g., via wired interconnects, wirelessly) to a controller 130. The controller 130 can include a microcontroller or a microprocessor that is configured to detect and track root growth.

As depicted in FIG. 1, controller 130 includes a communications unit (e.g., antenna 108, I/O port for cable 106) configured to transmit sensory data to a mobile device 154 (e.g., smart phone, tablet PC). In some instances, the communications unit can transmit sensory data over cable 106 to a mobile device 154. In some instances, cable 106 is a serial cable with appropriate connectors to interface with the communication unit of controller 130 and the mobile device 154. In such an instance, the communication unit includes circuitry (e.g., serial transceiver, etc.) to transmit and receive serial communications. In some examples, the communications unit can include an antenna 108 and circuitry configured to transmit sensory data wirelessly (e.g., Bluetooth, WiFi, or 900 MHz transmitter or antenna) to mobile device 154. In such an instance, the communication unit includes circuitry (e.g., Bluetooth transceiver, WiFi transceiver) to transmit and receive serial communications via wireless protocols. In some examples, the communications unit can include an antenna 108 and circuitry configured to transmit sensory data over a cellular network (e.g., 3G, 4G, LTE) to cellular tower or mobile device 154. In such an instance, the communication unit includes circuitry (e.g., 3G transceiver, 4G transceiver, LTE transceiver) to transmit and receive communications via cellular protocols.

The root phenotyping system 100 can also include one or more sensors (e.g., soil sensor 134, ambient sensor 136) associated with any desired aspect of plant 140, the soil location, and/or one or more above-ground conditions at or near the soil location. In general, the soil sensor 134 is located within the soil or at the air/soil interface, and the ambient sensor 136 is located above the soil or at the air/soil interface. For example, the soil sensor 134 can be configured to determine one or more nutrient levels (e.g., phosphorus, nitrogen, oxygen, soil humidity, temperature, moisture, pH, etc.) of the soil situated at or near the plant location. In some instances, soil sensor 134 is a nutrient sensor. In some instances, soil sensor 134 is a soil humidity sensor, a moisture sensor, or a temperature sensor.

The ambient sensor 136 is configured to determine one or more environmental/ambient conditions above ground. In some examples, the ambient sensor 136 is configured to determine one or more environmental conditions (e.g., humidity, temperature, light, etc.) associated with the plant. In some instances, the ambient sensor 136 is a temperature sensor or a humidity sensor. In some instances, the ambient sensor 136 is a rain sensor or a light sensor. Both the soil sensor 134 and the ambient sensor 136 provide in situ information regarding localized field locations (e.g., related to soil desiccation and/or fertilizer retention). This information assists breeders and growers in targeting irrigation and/or fertilizer to specific field locations, which provides cost and energy savings.

Power provided to controller 130 of the root phenotyping system 100 includes one or more power sources. For example, as depicted in FIG. 1, the root phenotyping system 100 can include solar cell 132 affixed to extended vertical support 114 to provide electrical power to controller 130. Other suitable power sources can include one or more solar cells, one or more batteries, or any combination thereof (e.g., solar cell 132 configured to charge a battery). In some examples, controller 130 of the present disclosure has both active and power-down modes, which provide for modulation of power consumption.

Additional details of the root phenotyping system 100 can be found in U.S. patent application Ser. No. 15/778,195, entitled “METHODS AND DEVICES FOR NON-INVASIVE ROOT PHENOTYPING,” filed May 22, 2018, the content of which is hereby incorporated by reference with regard to root phenotyping systems, as well as components and features thereof.

FIG. 2 illustrates a plurality of exemplary LED units configured to collect data for detecting root growth and root traits, according to some embodiments. As shown in FIG. 2, six LED units are affixed onto the surface of a paddle 220. The paddle 220 can be any of the paddles 126 shown in FIG. 1 and can be affixed to a support structure such as the cage structure 120 shown in FIG. 1. In some embodiments, the paddle may be water proof to prevent shorting. In some embodiments, waterproofing is achieved using clear epoxy resin potting and encapsulating material. The liquid resin is applied over the exposed solder connections to the emitter and detector units and hardens to form a clear plastic optical window. This layer covers the exposed electrical connections but may extend over more of the surface of the paddle to create a geometry that is more favorable to insertion into soil. The epoxy encapsulant is desirable for mechanical protection of the emitter and detector against the forces of inserting into the soil as well. A system of the present disclosure may comprise one or more, two or more, three or more, four or more, five or more, six or more, seven or more, eight or more, nine or more, ten or more, fifteen or more, or twenty or more paddles.

FIG. 2 further provides a magnified view of one of the six LED units. As shown, the LED unit 230 comprises an emitter 232 and a detector 234. The emitter 232 is configured to produce a plurality of outgoing light signals. The emitter can be implemented via any type of light source capable of producing light signals. In some embodiments, the emitter includes one or more LED lights of the same or different colors. In certain embodiments, the emitter is a red light, narrow band LED emitter and/or the detector is a broadband phototransistor detector.

The light signals produced by the emitter can be single-wavelength or multi-wavelength. If the emitter is narrow band (e.g., single wavelength) and multiple emitters with different wavelengths paired with a broad band detector, the color of the detected object can be determined by the relative difference between the signals produced by illumination by one color of light at a time. For example, if the object is red, it will produce a strong signal when illuminated with red light but a weak signal under green light. A white object would have a strong signal under both red and green illumination, and a green object would respond strongly to green illumination but weakly to red. If emitters are red, green, and blue, a reasonable color determination can be made with any color object. The narrow band emitter also may be lower-cost and involve simpler implementation. A broad band light source has the advantage of having better uniform response to all detected object colors. In some embodiments, color determination can be done with a single broad spectrum emitter paired with several narrow band detectors. The detected object would be illuminated with white light, and detectors sensitive to only a narrow band of reflected light can be used to determine the color of the object.

In some embodiments, the emitter of the LED unit is selected based on its spectral emission, luminous efficiency, or a combination thereof.

The detector 234 is configured to receive a plurality of returned light signals corresponding to the plurality of outgoing light signals. The detector 234 can be implemented via any type of detector capable of sensing presence and characteristics (e.g., magnitude) of light signals. In some embodiments, the detector includes one or more phototransistors and/or one or more photodiodes. The detector can be single-wavelength or multi-wavelength. The detector 234 can produce analog voltage readings in response to incoming light signals. In some embodiments, the detector of the LED unit is selected based on its spectral response, responsivity, dark current, response time, noise spectrum, or a combination thereof.

The LED unit 230 can be electrically coupled to a microprocessor (not depicted). In some embodiments, the analog voltage readings produced by the detector 234 are converted into digital readings by an analog-to-digital converter. The converter can be a part of the microprocessor. The microprocessor can adjust the settings of the converter to take readings using different reference voltages to change the range and precision of the readings.

In some embodiments, the LED unit is selected for the non-invasive root phenotyping system based on the soil type, the root type, or a combination thereof. The soil in different geographical locations may have different physical characteristics, such as color, density, and reflectivity. Further, different types of plant may have roots of different colors, sizes, and shapes. Thus, the color spectrum in LED unit may be adjusted based on the characteristics of the soil and the root. For example, and without wishing to be bound to theory, with a single-color detector unit the best signal response may come from a detector that is more sensitive to the color of the root than the color of the soil. For dark red clay soil, a white root may be best detected by a green light, as this would maximize the greatest difference between root and soil, as the soil would reflect green light poorly. Thus, the color spectrum in the LED unit could be selected to minimize reflection from soil and other objects while maximizing reflection from the plant root.

In some embodiments, the LED unit 230 further comprises a partition 236. The partition 236 is configured to prevent the detector from directly receiving and detecting the outgoing light signals produced by the emitter of the LED unit. The partition 236 can direct the outgoing light signal such that it travels toward the soil surface and gets reflected by the soil or the objects in the soil (e.g., roots) before reaching the detector. Thus, the partition can improve the performance of the root detection system by preventing cross-talking between the emitter 232 and the detector 234. In some embodiments, the partition is of a plastic material and manufactured using 3D printing technologies. In some embodiments, the partition is manufactured by casting or computer numeric control (CNC) tool.

In some embodiments, LED unit 230 further comprises a lens. The lens is configured to focus the incoming light onto the detector. In some embodiments, either the same or a second lens can be used to focus the light from the emitter toward the soil surface for more intense illumination. The lens can be of any shape, such as circular, square, or hexagonal.

In the depicted example, the six LED units are arranged in a linear configuration on the paddle 220. It should be appreciated that any number of LED units can be arranged in any configuration on the paddle. Depending on the expected growth pattern and physical characteristics of the root (e.g., length, angle, shape), the arrangement of the LED units and the relative positioning of the LED units to the plant can be made to facilitate detection of root growth. For example, the LED units can be arranged and positioned along the expected length of the root of the plant. As another example, the LED units can be arranged along the outer edge of the paddle, wherein most of the roots are expected to pass by. Further, multiple rows of LED units can be arranged on the paddle to facilitate detection of a close cluster of roots. In some embodiments, two or more rows are arranged on the paddle vertically or horizontally offset from each other. In some embodiments, the orientation and placement of the paddle and/or the support structure can be determined in a similar manner.

In some embodiments, one or more capacitive sensors (not depicted) are affixed to the paddle 220 in addition to the LED units. Capacitive touch sensors can be more suitable for detecting a root when the root is of a darker color or the contrast between the root and the soil is less visible. However, the performance of capacitive touch sensors can be negatively affected by certain environmental factors, such as electrical properties in the soil (water saturated and/or compacted). If the soil has high electrical conductivity either from being wet, salty, compacted or a combination of causes, the signal available from the capacitive touch sensors becomes weaker. Under these conditions, the reliability of the detection of capacitive sensors may be diminished. The higher the soil conductivity, the more signal is lost. This means few or no root touches can be detected until the soil dries and becomes less conductive. In contrast, LED units are generally less immune to environmental factors and can generate cleaner signals. Thus, LED units and capacitive sensors can be used concurrently to gather multiple sets of data which, when aggregated and cross-referenced, can produce more accurate results. As an example, a signal processing process for root detection using the capacitive touch system may involve comparison of an individual detector's signal to the signals of the detectors on the same paddle, to the signals of detectors on other paddles at the same depth in the soil, to the individual unit's global average signal, and to the average signals of other units (e.g., LED units) deployed at the same field site. Because there is noise in the capacitive touch detection system, these methods are needed to distinguish global events like a rainstorm from individual detector events like a root touch. In other words, additional information can be gleaned from the data set by making comparisons across many sensors (and many different types of sensors) to identify and correct for noise. Additional details on the use of capacitive touch sensors to detect root growth are provided in U.S. Provisional patent application Ser. No. 15/778,195, entitled “METHODS AND DEVICES FOR NON-INVASIVE ROOT PFIENOTYPING,” filed May 22, 2018, the content of which is hereby incorporated by reference with regard to root phenotyping systems, as well as components and features thereof.

In some embodiments, a system of the present disclosure may comprise one or more LED units of the present disclosure and one or more conductor plates (e.g., as described in U.S. patent application Ser. No. 15/778,195). Without wishing to be bound to theory, it is thought that LED units may be more suited for root detection in certain types of soil (e.g., water-saturated, compacted, etc.) and/or certain types of plant roots, as compared to conductor plates. The LED units and the capacitive touch sensors can be co-located or interspersed on the paddle. For example, a LED unit can be located in the middle of a capacitive detector pad. As another example, the LED units and the capacitive touch sensors can be arranged in a linear configuration in an alternating manner.

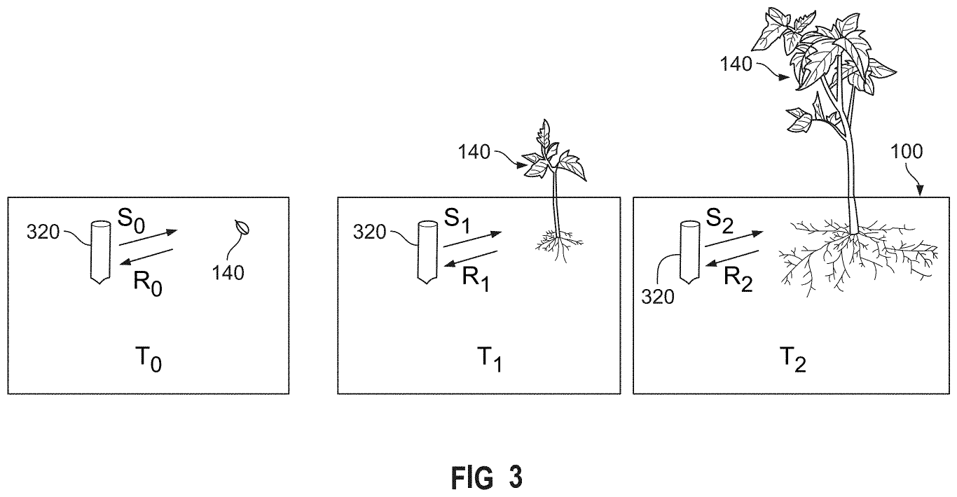

FIG. 3 illustrates an exemplary process for detecting root growth and traits using the present invention, in accordance with some embodiments. As shown, paddle 320 is vertically disposed in the soil. The paddle 320 includes at least one LED unit (not depicted) affixed onto the paddle. The LED unit can be the LED unit 230 described with reference to FIG. 2.

At T₀, T₁, and T₂, the emitter of the LED unit on the paddle 320 produces outgoing light signals S₀, S₁, and S₂, respectively. Subsequently, the detector of the LED unit receives the corresponding returned light signals R₀, R₁, and R₂, respectively. Each of the plurality of returned light signals R₀, R₁, and R₂ comprises at least a portion of the corresponding outgoing light signal S₀, S₁, and S₂ that is reflected from at least one of the soil and the root. In particular, R₀ comprises a portion of the outgoing light signal S₀ reflected from the soil (e.g., surface of the soil). R₁ comprises a portion of the outgoing light signal S₁ reflected from the soil and the root of plant 140. R₂ comprises a portion of the outgoing light signal S₂ reflected from the soil and the root of plant 140.

As the root of the plant 140 grows in the soil, the root can reflect a larger amount of light. For example, if the LED unit produces the same outgoing light signals at T₀, T₁, and T₂ (i.e., S₀, S₁, and S₂ are identical in magnitude and direction), the returned light signals R₀, R₁, and R₂ received by the LED unit are different in magnitude. Specifically, R₂ is higher than R₁ in magnitude, because the root of plant 140 at T₂ can reflect more light than the root at T₁. Similarly, R₁ is larger than R₀ in magnitude, because the root of plant 140 at T₁ can reflect more light than merely the soil at T₀.

As discussed above, the LED unit on the paddle 320 can be electrically coupled to a microprocessor. The microprocessor can process each returned light signal to obtain various characteristics of the light signal, such as the magnitude. By analyzing how characteristics of the light signals vary over time and by correlating the light signals with the locations of the LED unit(s) that detected the light signals, the microprocessor can obtain rich information about the objects and events in the soil. For example, returning light signals that become stronger (e.g., brighter) over time can indicate the presence of an object (e.g., appearance of a root) or the movement of an object (e.g., distal end of the root getting closer to the detector). As another example, for LED units with multiple emitters or detectors, color can be measured by the relative magnitude change when switching between different wavelengths.

The returning light signals can also be used to determine the type of object in the soil. An object in the soil can be relatively stationary (e.g., root) or dynamic (e.g., invertebrate). Based on how the returning light signals change over time and characteristics of the returning light signals, the system can determine whether the object is stationary or moving. The rate of magnitude change over time, as well as the permanence of the signal, can be used to help determine the type of object. Changes that occur very quickly are not likely to be root growth. A simple method would be analyzing the data to determine the time taken for the signal to go from a baseline reading to exceed a threshold. If that time scale is not reasonable for root growth, which should take several hours, it is likely not a root. A bug or worm would be more likely to take seconds or minutes to produce the same change in signal magnitude, Further, changes that go away after some time are also not likely to he roots. Thus, the system can look for signal decay after a potential detection. If the signal returns to its baseline level, it would be more likely to be the result of an invertebrate which moved away from the detector.

The returning light signals can also be used to determine the physical characteristics of the object in the soil. For example, the system can determine the angle of a root, the shape of a root, and the density of a group of roots. Further, the system can determine the rate of growth of a root.

The microprocessor can process the data received from the LED unit locally, or pass the data to a radio transmitter, which transmits the data wirelessly for storage and processing on a remote computer system. The remote computer system can receive data from multiple LED units, multiple paddles, and/or multiple cage structures. Further, the remote computer system can receive data from other types of sensors, such as soil sensors and ambient sensors. By aggregating data from different geographical locations and/or from different sensors, the system can obtain rich data on root growth patterns and how growth of roots are affected by different geographical locations, different soil conditions, and different environmental factors (e.g., presence of bacteria, invertebrate, etc.).

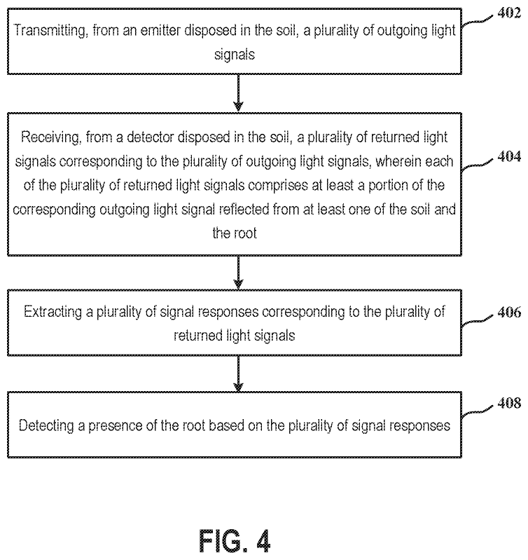

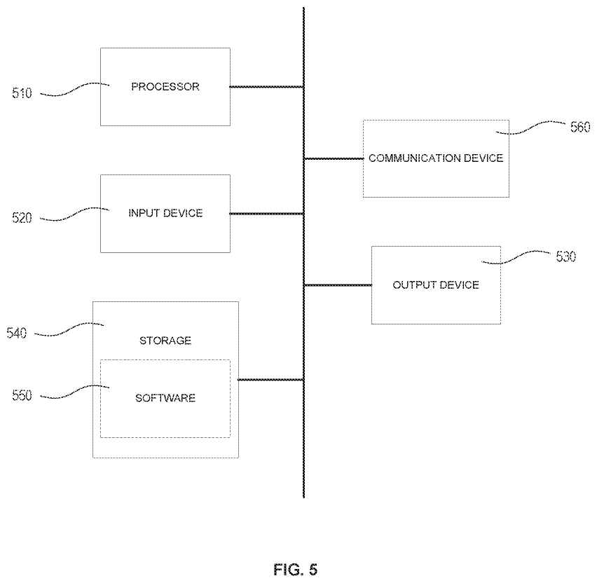

FIG. 4 depicts an exemplary process for detecting a root of a plant in the soil, in accordance with some embodiments. Process 400 is performed, for example, using one or more electronic devices. In some examples, the blocks of process 400 are divided up in any manner among the one or more electronic devices performing process 400. In some examples, the one or more electronic devices include the LED unit(s) disposed in the soil, the microprocessor(s) electrically coupled to the LED unit(s), the remote computer system, and/or additional electronic devices that are communicatively coupled with each other. Thus, while portions of process 400 are described herein as being performed by particular devices, it will be appreciated that process 400 is not so limited. In process 400, some blocks are, optionally, combined, the order of some blocks is, optionally, changed, and some blocks are, optionally, omitted. In some examples, additional steps may be performed in combination with the process 400. Accordingly, the operations as illustrated (and described in greater detail below) are exemplary by nature and, as such, should not be viewed as limiting.

At block 402, a plurality of outgoing light signals is transmitted from an emitter disposed in the soil. At bock 404, a detector disposed in the soil receives a plurality of returned light signals corresponding to the plurality of outgoing light signals. Each of the plurality of returned light signals comprises at least a portion of the corresponding outgoing light signal reflected from at least one of the soil and the root. At block 406, a plurality of signal responses corresponding to the plurality of returned light signals are extracted. At block 408, a presence of the root is detected based on the plurality of signal responses.

The operations described above with reference to FIG. 4 are optionally implemented by components depicted in FIG. 5. FIG. 5 illustrates an example of a computing device in accordance with one embodiment. Device 500 can be a host computer connected to a network. Device 500 can be a client computer or a server. As shown in FIG. 5, device 500 can be any suitable type of microprocessor-based device, such as a personal computer, workstation, server or handheld computing device (portable electronic device) such as a phone or tablet. The device can include, for example, one or more of processor 510, input device 520, output device 530, storage 540, and communication device 560. Input device 520 and output device 530 can generally correspond to those described above, and can either be connectable or integrated with the computer.

Input device 520 can be any suitable device that provides input, such as a touch screen, keyboard or keypad, mouse, or voice-recognition device. Output device 530 can be any suitable device that provides output, such as a touch screen, haptics device, or speaker.

Storage 540 can be any suitable device that provides storage, such as an electrical, magnetic or optical memory including a RAM, cache, hard drive, or removable storage disk. Communication device 560 can include any suitable device capable of transmitting and receiving signals over a network, such as a network interface chip or device. The components of the computer can be connected in any suitable manner, such as via a physical bus or wirelessly.

Software 550, which can be stored in storage 540 and executed by processor 510, can include, for example, the programming that embodies the functionality of the present disclosure (e.g., as embodied in the devices as described above)

Software 550 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 540, that can contain or store programming for use by or in connection with an instruction execution system, apparatus, or device.

Software 550 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 500 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 500 can implement any operating system suitable for operating on the network. Software 550 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

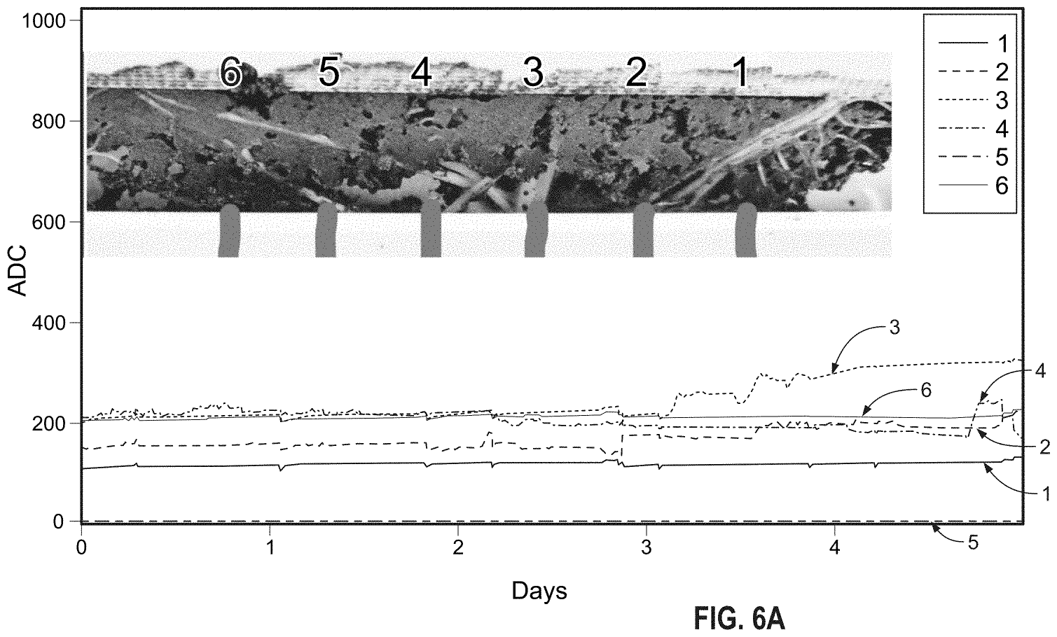

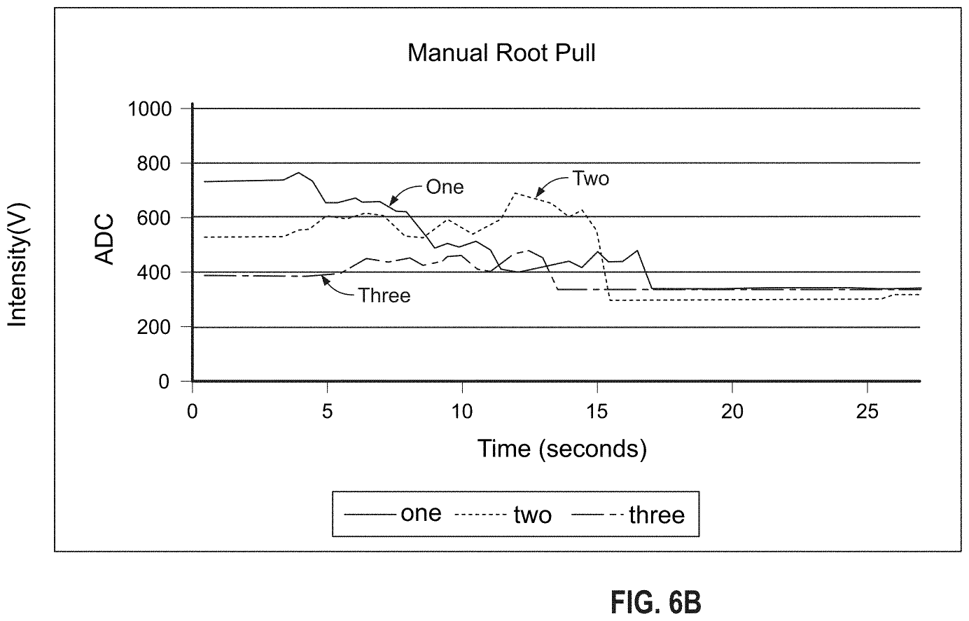

FIG. 6A depicts exemplary data related to root growth obtained by a plurality of exemplary LED units. In the depicted example, a paddle having six LED units was disposed horizontally in soil below a growing corn seed or corn plant. Red light, narrow band LED emitters and broadband phototransistor detectors were used. The LED units were evenly spaced on the paddle, as indicated by numerals 1, 2, 3, 4, 5, and 6 in the photo. The diagram shows the readings (using raw ADC count as the unit. Maximum count was 1023, corresponding to 3.3V based on the ADC configuration) by the six LED units over a few days. As shown, a few LED units (e.g., LED unit 3) received more intense signals over time, indicating root growth in the soil at the corresponding locations, as confirmed by imaging a cross-section of the soil (image provided in FIG. 6A),

FIG. 6B depicts exemplary data related to root growth obtained by a plurality of exemplary LED units. In the depicted example, a paddle having three LED units was placed under a piece of paper with a bundle of roots in front of the paddle. The bundle of roots was slowly pulled from left to right across the detectors of the paddle, and intensity was measured as a function of time. Increased signal was observed when roots were present in front of the detector, and the signal varied as more or less roots were present directly in front of the detectors as the bundle was moved. Detector 3 was located on the end from which the roots were pulled away, and the signal returned to baseline first on detector 3, then detectors 2 and 1 in the expected order tracking movement of the bundle. These results demonstrate that the LED paddle was able to track the movement of the root bundle by measuring intensity of detected light.

The systems of the present disclosure may find use for a variety of applications. For example, a system of the present disclosure (e.g., comprising one or more paddles of the present disclosure) can be used to detect the presence of a plant root in soil, monitor growth of a plant root, detect a soil organism, select a plant for breeding, determine an effect of a plant-microbe interaction on a root growth characteristic, and/or determine an effect of soil composition and/or condition on a root growth characteristic.

In some embodiments, a system of the present disclosure (e.g., comprising one or more paddles of the present disclosure) is used in a method of detecting the presence of a plant root in soil. As exemplified in FIG. 6, the systems described herein are able to detect the presence of a root, as distinguished from the surrounded soil, via LED-based detection.

In some embodiments, a system of the present disclosure (e.g., comprising one or more paddles of the present disclosure) is used in a method of monitoring growth of a plant root in soil. For example, one or more paddles may be placed into the soil surrounding a plant and arranged so as to detect the growth of one or more roots of the plant. Alternatively, one or more paddles may be placed into the soil around a seed, or a location into which a seed is later planted, such that when the seed germinates and the plant roots begin to grow, the paddle(s) are arranged so as to detect the growth of one or more roots of the plant. In some embodiments, growth is monitored by successive detections of the plant root at one or more LEDs. For example, a plant root growing in front of LED unit A at time t1 is detected by LED unit A. If at time t2 the plant root is detected in front of LED unit A and LED unit B (e.g., on the same or a different paddle than LED unit A), and LED unit B is spaced at x distance from LED unit A, this indicates that the plant root grew across x distance between t1 and t2, and therefore a rate of growth and a root length can be calculated. Alternatively, if a seed is planted at time t1, and the root is detected at time t2, a rate of growth or establishment rate can be calculated as x distance over the interval between t1 and t2. Detected signals can be from thicker roots or more numerous roots (e.g., increased root density), both of which are indicative of increased root biomass. In addition, a root angle can be calculated by taking into account relative positions of LED units A and B. Through detection of plant root(s) by multiple LED units arranged in two or three dimensions (e.g., by factoring number and/or magnitude of detected signal(s), the time scale over which signal(s) were detected, and optionally inferred root angle), the length, number, and/or girth of plant root(s) can be calculated to estimate or inter total root biomass.

In some embodiments, a system of the present disclosure (e.g., comprising one or more paddles of the present disclosure) is used in a method of monitoring plant health or plant root health. For example, the detector can be configured to distinguish a change in color of the root (e.g., by distinguishing a white root from a brown root), which can indicate a change in root health or death of a root.

In some embodiments, a system of the present disclosure (e.g., comprising one or more paddles of the present disclosure) is used in a method of selecting a plant for breeding. For example, one or more root growth characteristics may be monitored (e.g., as described above) in order to assay one or more traits of interest, e.g., related to root properties. Using the systems of the present disclosure, one or more replicates of a plant variety/line can be monitored for root growth characteristic(s) of interest, and a variety or line of plants can be selected for breeding based on one or more of the root growth characteristics of the present disclosure. Such plants can be crossed with a plant of the same species or variety, or a different species or variety (for hybrids) to produce progeny, thereby successfully breeding the plant for a trait or characteristic of interest.

In some embodiments, a system of the present disclosure (e.g., comprising one or more paddles of the present disclosure) is used in a method of determining an effect of a plant-microbe interaction on a root growth characteristic. One or more root growth characteristics may be monitored (e.g., as described above) using a system of the present disclosure in order to assay the effect of a plant-microbe interaction. For example, root growth of a plant grown in soil inoculated with a microbe or community of microbes can be compared against root growth of a plant grown in soil inoculated with a different microbe or community of microbes, or without the microbe or community of microbes, or with another suitable reference. In some embodiments, one or more aspects of a plant rhizosphere can be studied by tracking root growth with a system of the present disclosure.

In some embodiments, a system of the present disclosure (e.g., comprising one or more paddles of the present disclosure) is used in a method of determining an effect of soil composition and/or condition on a root growth characteristic. This allows the determination of how plant roots change growth/behavior based on soil conditions/composition (e.g., presence or absence of one or more nutrients, water conditions, soil packing, aeration, etc.). One or more root growth characteristics may be monitored (e.g., as described above) using a system of the present disclosure in order to assay the effect of soil composition and/or condition. For example, root growth of a plant grown in soil having a particular composition/condition can be compared against root growth of a plant grown in soil having a different composition/condition, or with another suitable reference plant.

The systems of the present disclosure may be used (e.g., as in the methods described above) to detect roots of a variety of plants. In some embodiments, the plant is a row crop. In some embodiments, the plant is a commercially grown plant, such as maize, soybean, rice, wheat, sorghum, tomato, or alfalfa.

In some embodiments, a system of the present disclosure (e.g., comprising one or more paddles of the present disclosure) is used in a method of detecting a soil organism. It is thought that a soil organism can be detected by received light signals as described for a plant root herein. However, since plant roots are more stationary than a soil organism, a more transient signal at one or more LED units can indicate the presence of a moving organism, rather than a plant root. In some embodiments, a color of the organism may be detected. In some embodiments, the soil organism is a worm or insect. In some embodiments, the soil organism is a corn root worm.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

1. A system for detecting a root of a plant in soil, the system comprising:
a support structure configured to be at least partially disposed in the soil;
an LED unit affixed to the support structure,
wherein the LED unit comprises an emitter and a detector,
wherein the emitter is configured to produce a plurality of outgoing light signals,
wherein the detector is configured to receive a plurality of returned light signals corresponding to the plurality of outgoing light signals, and
wherein each of the plurality of returned light signals comprises at least a portion of the corresponding outgoing light signal reflected from at least one of the soil and the root; and
a microprocessor configured to detect a presence of the root based on the plurality of returned light signals.

2. The system of claim 1, further comprising a signal extractor configured to extract a plurality of digital readings based on the plurality of returned light signals.

3. The system of claim 2, wherein the signal extractor comprises a voltage divider, an analog-to-digital converter, or a combination thereof.

4. The system of claim 1, wherein detecting the root based on the plurality of returned light signals comprises: determining a difference between a brightness of a first returned signal and a brightness of a second returned signal of the plurality of returned signals.

5. The system of claim 1, wherein the LED unit further comprises a partition, wherein the partition is configured to reduce detection of the plurality of outgoing light signals by the detector.

6. The system of claim 1, wherein the LED unit further comprises a lens.

7. (canceled)

8. The system of claim 1, wherein the support structure comprises a paddle, wherein the paddle comprises a plurality of LED units affixed thereto.

9. The system of claim 8, wherein the plurality of LED units are arranged in a linear configuration, or are arranged based on one or more characteristics of the plant.

10. (canceled)

11. The system of claim 1, further comprising one or more capacitive sensors for detecting the root of the plant.

12. The system of claim 1, wherein information associated with the plurality of returned light signals is transmitted to a remote computer system via a wireless network.

13. The system of claim 1, wherein the microprocessor is configured to detect a presence of an invertebrate in the soil based on the plurality of returned light signals.

14. The system of claim 1, wherein the microprocessor is configured to determine, based on the plurality of returned light signals: a growth rate of the root, an angle of the root, a density of a group of roots, or a combination thereof.

15. The system of claim 1, further comprising a power supply electrically coupled to the LED unit, wherein the power supply is configured to provide an electrical charge to the LED unit.

16. A method for detecting a root of a plant in soil, the method comprising:
transmitting, from an emitter disposed in the soil, a plurality of outgoing light signals;
receiving, from a detector disposed in the soil, a plurality of returned light signals corresponding to the plurality of outgoing light signals, wherein each of the plurality of returned light signals comprises at least a portion of the corresponding outgoing light signal reflected from at least one of the soil and the root;
extracting a plurality of signal responses corresponding to the plurality of returned light signals; and
detecting a presence of the root based on the plurality of signal responses.

17. (canceled)

18. The method of claim 16, further comprising:
transmitting, from a second emitter disposed in the soil, a second plurality of outgoing light signals;
receiving, from a second detector disposed in the soil, a second plurality of returned light signals corresponding to the second plurality of outgoing light signals, wherein each of the second plurality of returned light signals comprises at least a portion of the corresponding outgoing light signal reflected from at least one of the soil and the root;
extracting a second plurality of signal responses corresponding to the second plurality of returned light signals;
detecting a presence of the root based on the second plurality of signal responses;
based on the first and the second plurality of signal responses, determining a growth characteristic of the plant root, wherein the growth characteristic is selected from the group consisting of growth rate, root angle, root length, and root biomass.

19. The method of claim 16, wherein the plant is a row crop or is selected from the group consisting of maize, soybean, rice, wheat, sorghum, tomato, and alfalfa.

20. (canceled)

21. A method for detecting a soil organism, the method comprising:
transmitting, from an emitter disposed in the soil, a plurality of outgoing light signals;
receiving, from a detector disposed in the soil, a plurality of returned light signals corresponding to the plurality of outgoing light signals, wherein each of the plurality of returned light signals comprises at least a portion of the corresponding outgoing light signal reflected from at least one of the soil and the soil organism;
extracting a plurality of signal responses corresponding to the plurality of returned light signals; and
detecting a presence of the soil organism based on the plurality of signal responses.

22. (canceled)

23. The method of claim 21, wherein the soil organism is a worm or insect.

24. (canceled)

25. A method for monitoring growth of a root of a plant in soil, the method comprising:
positioning a plurality of emitters and a plurality of detectors around a soil location, wherein a plant having a root is planted in the soil location;
transmitting, from an emitter of the plurality of emitters disposed in the soil, a plurality of outgoing light signals;
receiving, from a detector of the plurality of detectors disposed in the soil, a plurality of returned light signals corresponding to the plurality of outgoing light signals, wherein each of the plurality of returned light signals comprises at least a portion of the corresponding outgoing light signal reflected from at least one of the soil and the root;
extracting a plurality of signal responses corresponding to the plurality of returned light signals;
detecting a presence of the root based on the plurality of signal responses; and
determining a growth characteristic of the plant root based on the detected presence of the root.

26. A method for monitoring growth of a root of a plant in soil, the method comprising:
planting a seed in a soil location;
positioning a plurality of emitters and a plurality of detectors around the soil location;
after the seed has grown into a plant having a root, transmitting, from an emitter of the plurality of emitters disposed in the soil, a plurality of outgoing light signals;
receiving, from a detector of the plurality of detectors disposed in the soil, a plurality of returned light signals corresponding to the plurality of outgoing light signals, wherein each of the plurality of returned light signals comprises at least a portion of the corresponding outgoing light signal reflected from at least one of the soil and the root;
extracting a plurality of signal responses corresponding to the plurality of returned light signals;
detecting a presence of the root based on the plurality of signal responses; and
determining a growth characteristic of the plant root based on the detected presence of the root.
